(19) United States
(12) Patent Application Publication
(10) Pub. No.: US 2009/0131904 A1
(43) Pub. Date: May 21, 2009

(54) INTERNAL THREADS IN TUBING

(76) Inventors: John D. Wright, Wyoming, MN (US); Charles M. Berg, Forest Lake, MN (US); Robert Palme, Lindstrom, MN (US); John W. Warling, Maplewood, MN (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/942,614

(22) Filed: Nov. 19, 2007

(51) Int. Cl. F16L 9/00; B23P 11/00; B23K 26/00; B23K 15/00; B23K 31/02; A61M 31/00; A61B 17/00

(52) U.S. Cl. 604/500; 138/177; 29/428; 219/121.64; 219/121.14; 128/898; 606/1

(57) ABSTRACT

Embodiments of the present invention provide an internally threaded tube of virtually limitless length that can be easily and reliably constructed. In one aspect, the invention provides an internally threaded tube that includes a tube casing and a coil. A ratio of the length of the tube casing to the inner diameter of the tube casing can be greater than 5:1. The coil can be positioned coaxially within the tube casing. In this position, the coil can exert a radially outward force on the inner surface of the tube casing, which can aid in bonding. A portion of the coil can be specially adapted to be bonded to the tube casing. Methods of creating internally threaded tubes and methods of spirally delivering surgical components with internally threaded tubes are also disclosed.

### TECHNICAL FIELD

This disclosure is related to tubing having internal threads.

### BACKGROUND

Forming internal threads can be a difficult process. Conventional methods involve cutting threads into a casing with a tap. Such methods pose a variety of limitations, especially as the length of the tubing increases. For example, keeping the tap from wandering off center can be difficult, if not impossible, for longer casings. Also, as the length of the casing increases, it becomes more difficult to remove cut material from the interior of the casing while the tap is cutting the threads. Additionally, in many instances, a counter bore is required. Aligning the counter bore becomes significantly more difficult as the casing length increases. These difficulties can make such conventional methods impractical, if not impossible, for many applications.

### SUMMARY

Embodiments of the present invention provide an internally threaded tube of virtually limitless length that can be easily and reliably constructed. In one aspect, the invention provides an internally threaded tube that includes a tube casing and a coil. The tube casing can have inner and outer surfaces. The inner surface can have a substantially circular cross-sectional profile. A ratio of the length of the tube casing to the inner diameter of the tube casing can be greater than 5:1. The coil can be positioned coaxially within the tube casing. In this position, the coil can exert a radially outward force on the inner surface of the tube casing. The coil can comprise an elongate element that is formed into a generally helical shape. A first portion of the element can interface with the inner surface of the tube casing. A second portion of the element can project inwardly to form internal threads. The first portion of the element can be specially adapted to be bonded to the tube casing. The coil can be bonded to the tube casing at one or more sites along the interface of the first portion of the element and the inner surface of the tube casing.

In a second aspect, the invention provides a method of creating an internally threaded tube. The method can include providing a tube casing and a coil. The method can also include positioning the coil coaxially within the tube casing such that the first portion of the element interfaces with the inner surface of the tube casing. In this position, the coil can exert a radially outward force on the inner surface of the tube casing. The method can further include bonding the coil to the tube casing at one or more sites along the interface of the first portion of the element and the inner surface of the tube casing. In this position, a second portion of the element can project inwardly to form internal threads.

In a third aspect, the invention provides a method of spirally delivering a surgical component to internal tissue. The method can include providing an internally threaded tube and positioning a distal end of that tube proximate to internal tissue. The method can also include spirally delivering a surgical component from a proximal end of the internally threaded tube through the distal end of the internally threaded tube to the internal tissue.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a perspective view of an internally threaded tube, according to some embodiments of the present invention.

FIG. 2A is a side plan view of a coil of the internally threaded tube of FIG. 1 before assembly.

FIG. 2B is a side plan view of a tube casing of the internally threaded tube of FIG. 1 before assembly.

FIG. 3 is a more detailed view of a portion (detail B) of FIG. 2A.

FIG. 4 is a cross-sectional view (section A-A) of a portion of the internally threaded tube of FIG. 1.

FIG. 5 is a more detailed view of a portion (detail B) of FIG. 5.

FIG. 6 is a more detailed view of a portion (detail B) of FIG. 5 with a laser beam operating on the internally threaded tube.

FIG. 7A is an end view of a laser welding apparatus and an internally threaded coil, according to some embodiments of the present invention.

FIG. 7B is a cross-sectional view (section A-A) of the laser welding apparatus and the internally threaded coil of FIG. 7A.

FIG. 8A is a side plan view of a fixture that can be used in some embodiments of the present invention.

FIG. 8B is a cross-sectional view (section A-A) of a portion of the fixture of FIG. 8A.

FIG. 9 is a schematic view of a surgical component being spirally delivered to internal tissue, according to some embodiments of the present invention.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Examples of constructions, materials, dimensions, and manufacturing processes are provided for selected elements, and all other elements employ that which is known to those of skill in the field of the invention. Those skilled in the art will recognize that many of the examples provided have suitable alternatives that can be utilized.

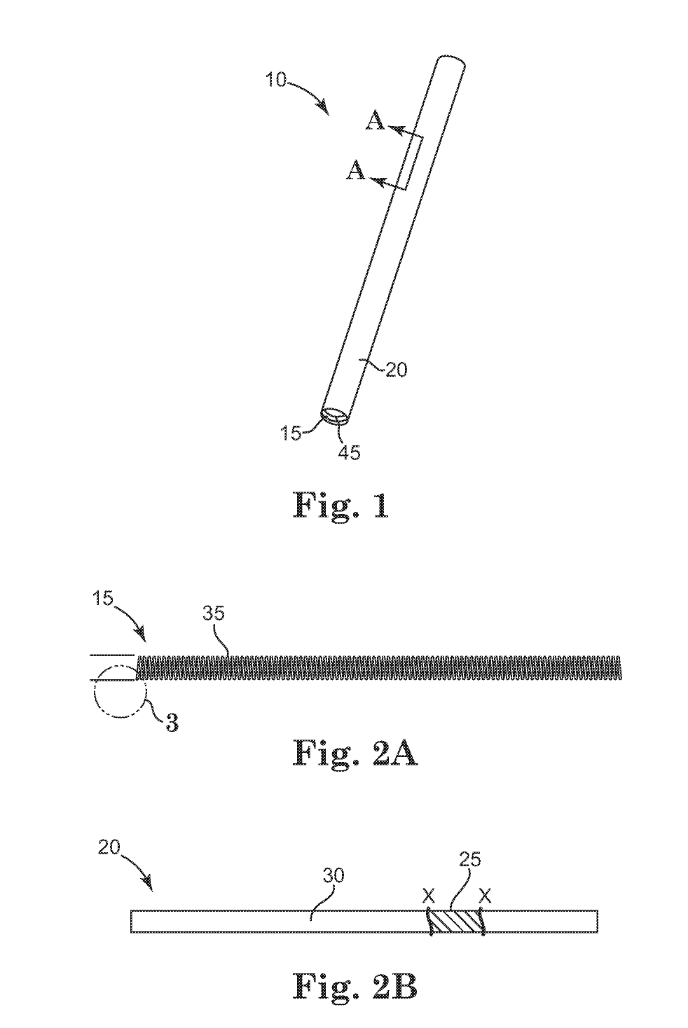

FIG. 1 shows an internally threaded tube 10, according to embodiments of the present invention. The internally threaded tube 10 can include a coil 15 positioned coaxially within a tube casing 20. The coil 15 and the tube casing 20 can be bonded together, with a portion of the coil 15 extending inwardly to form internal threads. Internally threaded tubes configured according to embodiments of the present invention can be used in a variety of applications, such as spirally delivering surgical components (e.g., fixation helices, tacks, screws, fasteners or various spiral wound fixation devices) to internal tissue.

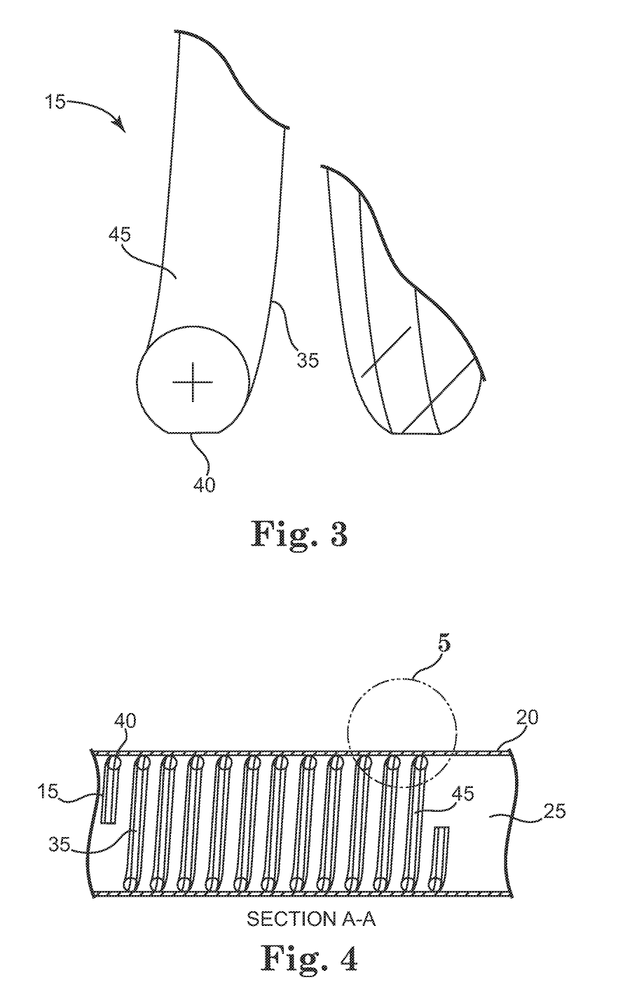

FIG. 2B shows a tube casing 20 that can be used in some embodiments of the present invention. Tube casings can have a variety of attributes. Many tube casings are made of 300- and 400-series stainless steel, titanium, monel, mp35, hasteloy and various members of the stainless steel family. In many embodiments, the tube casing 20 is biocompatible. As shown, the tube casing 20 can have an inner surface 25 (shown via cutaway X-X) and an outer surface 30. The inner surface 25 of the tube casing 20 often has a substantially circular cross-sectional profile. In many embodiments, the outer surface 30 has a substantially circular cross-sectional profile. Other cross-sectional profiles are possible, such as polygonal, elliptical, and other suitable cross-sectional profiles. Most tube casings are unitary. Most tube casings are integrally formed.

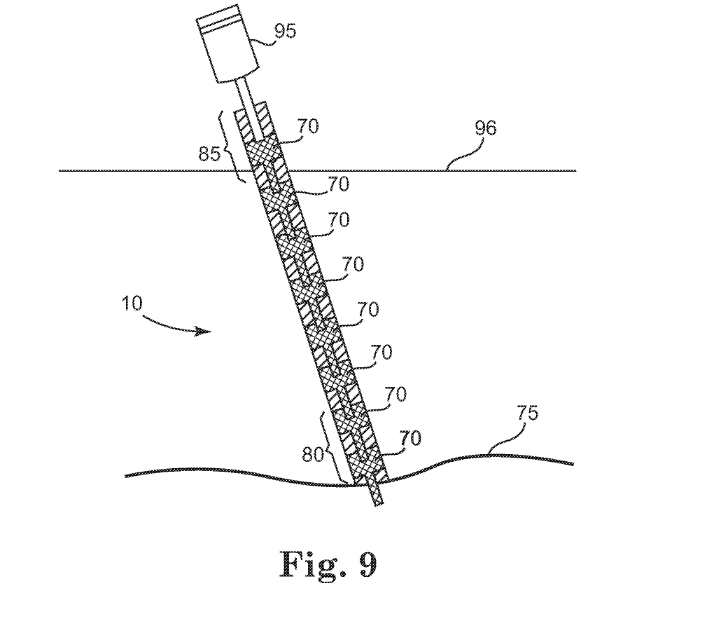

The tube casing 20 is often quite long in relation to the inner diameter of the tube casing 20. For example, when spirally delivering surgical components to internal tissue, the tube casing 20 should extend from the exterior of the surgical patient all the way into the patient's body to a position proximate to the relevant internal tissue (to be discussed in greater detail in connection with FIG. 9). In most embodiments, a ratio of the length of the tube casing 20 to the inner diameter of the tube casing 20 is greater than 5:1. In some preferred embodiments, that ratio is greater than 10:1. In some particularly preferred embodiments, that ratio is greater than 15:1. In most embodiments, the length of the tube casing 20 is greater than one inch. In some preferred embodiments, the length of the tube casing 20 is between three and five inches. In some particularly preferred embodiments, the length of the tube casing 20 is approximately four inches. In most embodiments, the inner diameter of the tube casing 20 is greater than ⅛ inch. In some preferred embodiments, the inner diameter of the tube casing 20 is between ⅛ inch and ½ inch. In some particularly preferred embodiments, the inner diameter of the tube casing 20 is approximately ⅕ inch.

In many instances, the length of the tube casing 20 and the internally threaded tube 10 is a function of the number of surgical components to be delivered by the tube 10 and the column height of each surgical component. For example, an application that requires 15 fasteners each having a column height of ¼ inch can be used in connection with a tube casing 20 and an internally threaded tube 10 that is 3¾ inches long. In another example, an application that requires 20 fasteners each having a column height of ¼ inch can be used in connection with a tube casing 20 and an internally threaded tube 10 that is 5 inches long. Tube casings of these lengths are nearly impossible to machine with a tap.

FIG. 2A shows a coil 15 that can be used in some embodiments of the present invention. A wide variety of coils can be used, depending on such factors as desired pitch, desired pitch depth, desired length, desired inner/outer diameters, the need for biocompatibility, and so on. The coil 15 can be made of any of the materials listed in connection with the tube casing, with 302- and 304-series stainless steel coils being most common. In most embodiments, the coil 15 can comprise an elongate element 35 formed into a generally helical shape. In many embodiments, the element 35 of the coil 15 is generally cylindrical. In some embodiments, the element 35 can have other cross-sectional profiles, such as a D-shape or a triangle. The ratio of the outer diameter of the coil 15 to the pitch is commonly similar to UNC and UNF ratios and most commonly between 4:1 and 8:1. In certain preferred embodiments of the present invention, the coil 15 can have a pitch of approximately 1/24 inch. In some embodiments, the internal threads formed by the coil 15 are adapted to mate with a threaded object having a minor diameter of approximately 0.19 inches.

FIG. 3 shows an example of how a first portion 40 of the coil 15 can be specially adapted to be bonded to the tube casing. As shown, the coil 15 is formed from a generally cylindrical element 35, with the cross-sectional profile of the first portion 40 of the element 35 being less curved than that of the second portion 45 of the element 35 (e.g., the cross-sectional profile of the first portion 40 can be substantially flat). In some embodiments, the coil 15 can be centerless ground so that the interface of the first portion 40 of the element 35 and the inner surface of the tube casing has increased surface contact, as compared with a similar coil that is not centerless ground. In embodiments in which the coil element 35 has a D-shaped or triangular cross-sectional profile, the cross-sectional profile of the first portion 40 can be substantially flat. In such embodiments, the first portion 40 of the element 35 can be specially adapted to be bonded to the tube casing even if the coil 15 is unmodified after the element is formed into a generally helical shape.

If a coil 15 formed by a cylindrical element 35 is not specially adapted to be bonded to the tube casing, bonding the coil 15 to the tube casing can be difficult. If the only interface between the coil 15 and the tube casing is the outermost edge of each coil revolution, trying to laser weld along that interface can result in blow holes, decreased weld joint quality/strength, and a host of additional contamination issues. In preferred embodiments, the surface contact between the first portion 40 of the element 35 and the tube casing permits a laser weld focal point to create bonds without encountering any air gaps between the coil 15 and the tube casing.

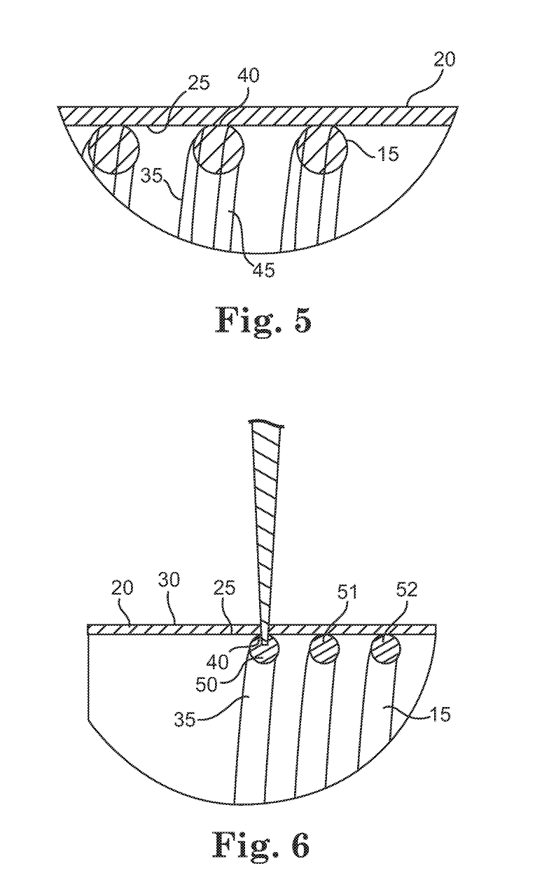

FIGS. 4-5 show the coil 15 positioned coaxially within the tube casing 20. In many instances, the length of the coil 15 can be substantially equal to the length of the tube casing 20. In some embodiments, a segment of the coil 15 can be removed near the end of the method for creating an internally threaded tube 10, thereby making sure that no part of the coil 15 is not positioned coaxially within the tube casing 20. A first portion 40 of the coil element 35 can interface with the inner surface 25 of the tube casing 20, and a second portion 45 of the coil element 35 can project inwardly to form internal threads. In this position, the coil 15 can exert a radially outward force on the inner surface 25 of the tube casing 20. This radially outward force can result from the coil 15 being compressed when positioned coaxially within the tube casing 20. In many embodiments, before assembly, the outer diameter of the coil 15 can be equal to or greater than the inner diameter of the tube casing 20. In many instances, the outer diameter of the coil 15 is approximately 0.002-0.007 inches greater than the inner diameter of the tube casing 20. The resulting radially outward force after assembly can create friction between the coil 15 and the tube casing 20, which aids in maintaining proper alignment and positioning. This force can eliminate any gap between the coil 15 and the tube casing, which can significantly reduce the incidence of blow holes during bonding.

In many cases, the length of the coil 15 before assembly is slightly less than the length of the tube casing 20. When the coil 15 is compressed and positioned coaxially within the tube casing 20, the length of the coil 15 can be increased (e.g., so that the length of the assembled coil and tube casing 20 are substantially equal). For example, a coil having a free state outer diameter of 0.205 inches can increase in length by approximately 0.024 coils for each compressed coil revolution when inserted into a tube casing having an inner diameter of 0.200 inches. Thus, according to this example, a coil having 100 coil revolutions would increase in length by approximately 2.4 coils. The interrelationship between the coil 15 and the tube casing 20 can depend on a variety of factors, such as index ratio and material elasticity.

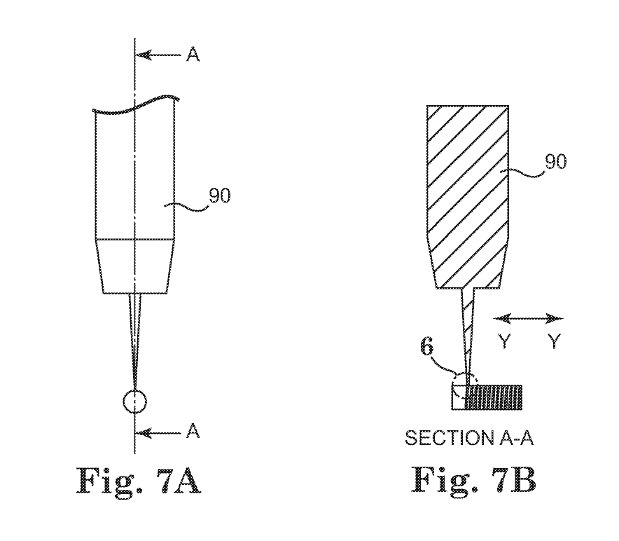

FIGS. 6, 7A, 7B show how the coil 15 can be bonded to the tube casing 20 at one or more sites along the interface of the first portion 40 of the element 35 and the inner surface 25 of the tube casing 20. In some preferred embodiments, the coil 15 is laser welded to the tube casing 20 at one or more sites along the interface of the first portion 40 of the element 35 and the inner surface 25 of the tube casing 20. In some such embodiments, one or more selected individual coil revolutions 50 can be bonded to the tube casing 20, while other coil revolutions 51-52 can remain un-bonded. For example, a first coil revolution can be laser welded to the tube casing 20, second through eighth coil revolutions can be un-bonded, a ninth coil revolution can be laser welded to the tube casing 20, tenth through sixteenth coil revolutions can be un-bonded, and a seventeenth coil revolution can be laser welded to the tube casing 20. Many suitable combinations are possible depending on application, friction between the coil 15 and the tube casing 20, strength of each bond site, length of the tube casing 20 and/or the coil 15, and so on. Methods of bonding the coil 15 to the tube casing 20 are discussed in greater detail below.

Embodiments of the present invention provide a method of creating an internally threaded tube. In some embodiments, the method includes providing a tube casing and a coil (e.g., like the tube casing 20 and coil 15 embodiments discussed above), positioning the coil coaxially within the tube casing, and bonding the coil to the tube casing. In this way, the coil can form internal threads in the tube.

As is discussed above, the coil 15 can be positioned coaxially within the tube casing 20. In this position, a first portion 40 of the element 35 can interface with the inner surface 25 of the tube casing 20. Because, in many embodiments, the outer diameter of the coil 15 is equal to or greater than the inner diameter of the tube casing 20, the coil 15 can exert a radially outward force on the inner surface 25 of the tube casing 20.

The coil 15 can be bonded to the tube casing 20 in a variety of ways (e.g., laser welding, adhesive, etc.). In many embodiments, bonding the coil 15 to the tube casing 20 can comprise directing a high-energy beam (e.g., with laser welder go of FIGS. 7A-7B) from the outer surface 30 of the tube casing 20 radially inwardly to bond selected individual coil revolutions to the tube casing 20. In some embodiments, laser welding can comprise subjecting the outer surface 30 of the tube casing 20 to a laser weld with a laser having a focal point diameter approximately 0.003 inches less than the width of the interface of the first portion 40 of the element 35 and the inner surface 25 of the tube casing 20. In some preferred embodiments, the width of the interface of the first portion 40 of the element 35 and the inner surface 25 of the tube casing 20 is approximately 0.009 inches, and the laser focal point diameter is approximately 0.007-0.008 inches.

Referring again to FIGS. 6, 7A, 7B, in some embodiments, selected individual coil revolutions can be bonded to the tube casing 20, while other coil revolutions can remain un-bonded. For example, bonding can include positioning a laser welder go proximate to the outer surface 30 of the tube casing 20, laser welding a first coil revolution of the coil 15 to the tube casing 20 at a first site, translating the laser welder longitudinally (e.g., along path Y-Y) along the outer surface 30 of the tube casing 20 past a first predetermined number (e.g., seven) of coil revolutions, laser welding a second coil revolution of the coil 15 to the tube casing 20 at a second site, translating the laser welder longitudinally (e.g., along path Y-Y) along the outer surface 30 of the tube casing 20 past a second predetermined number (e.g., seven) of coil revolutions, and laser welding a third coil revolution of the coil 15 to the tube casing 20 at a third site. In this example, every eighth coil revolution is bonded to the tube casing 20. In embodiments of the present invention, different increments of coil revolutions can be bonded to the tube casing 20, such as every other, every third, every fourth, and so on. In some embodiments, the bond sites run generally in a line. In some embodiments, the bond sites are spaced about the perimeter of the outer surface 30 of the tube casing 20. In some embodiments, a single bonding site can stretch substantially the entire length of the interface of the coil 15 and the tube casing 20. Embodiments of the present invention involve a variety of patterns and approaches for bonding the coil 15 to the tube casing 20. The patterns and approaches discussed herein are illustrative.

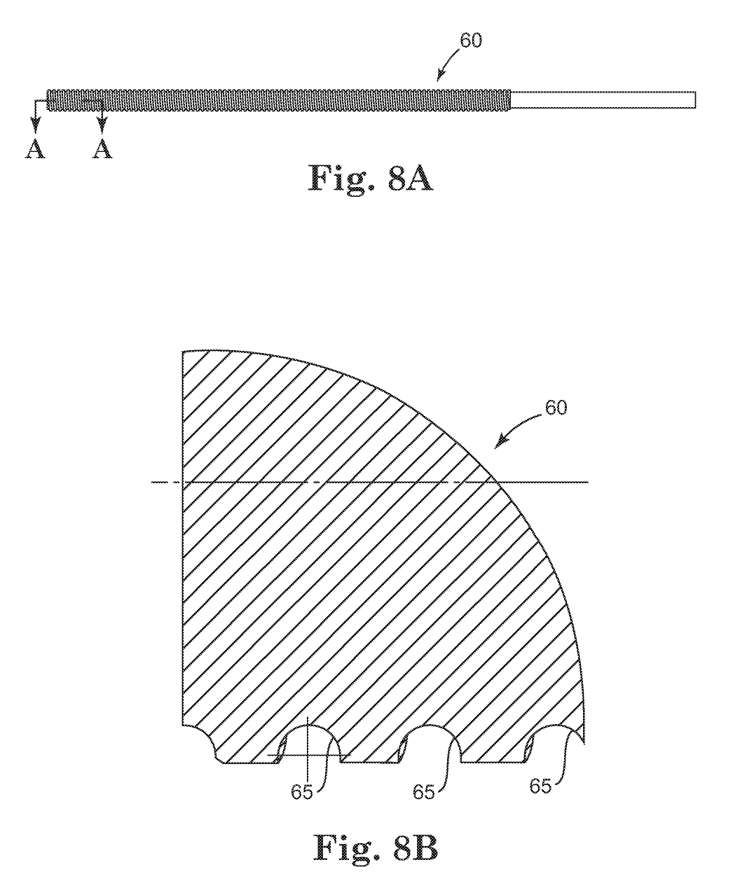

FIGS. 8A-8B show a fixture 60 (e.g., a threaded rod) that can be used to stabilize the coil while being positioned coaxially within and bonded to the tube casing 20, according to some embodiments of the present invention. The fixture 60 can be positioned coaxially within the coil. The fixture can have threads 65 with a pitch that is complementary with a pitch of the coil. When the fixture 60 has been positioned coaxially within the coil, both the coil and the fixture 60 can be positioned coaxially within the tube casing. In some embodiments, threads 65 of the fixture 60 are deep enough to permit the coil to deflect radially inwardly while the coil and the fixture 60 are being positioned coaxially within the tube casing. This feature can assist in accommodating a situation in which the outer diameter of the coil 15 is greater than or equal to the inner diameter of the tube casing. In some embodiments, a laser welder can be programmed to laser weld the coil to the tube casing based on the pitch of the threads 65 of the fixture 60. After the coil is bonded to the tube casing to form an internally threaded tube, the fixture 60 can be unscrewed from the internally threaded tube.

FIG. 9 shows multiple surgical components 70 being spirally delivered to internal tissue 75 (e.g., soft tissue, hard tissue, etc.). An internally threaded tube 10 can be provided. A distal end 80 of the internally threaded tube 10 can be positioned proximate to internal tissue 75. A proximal end 85 of the internally threaded tube 10 can be positioned proximate to the patient's skin 96.

One or more surgical components 70 can be spirally delivered from the proximal end 85 of the internally threaded tube 10 through the distal end 80 of the internally threaded tube 10 to the internal tissue 75. In some embodiments, a plurality of surgical components 70 can be spirally loaded into the internally threaded tube 10. In some such embodiments, spirally delivering a surgical component 70 comprises spirally driving a surgical component 70 nearest the proximal end 85 of the internally threaded tube 10 with an instrument 95, thereby causing a surgical component 70 nearest the distal end 80 of the internally threaded tube 10 to be spirally delivered to the internal tissue 75. For example, each surgical component 70 can have a male projection at its distal end and a complementary female receptacle at its proximal end. If a first surgical component 70 is positioned proximally of a second surgical component 70, the male projection of the first surgical component 70 can mate with the female receptacle of the second surgical component 70. When rotational force is applied to the female receptacle of the first surgical component 70 (e.g., by an instrument 95 or by the male projection of a different surgical component 70) the male projection of the first surgical component 70 can transfer that rotational force to the female receptacle of the second surgical component 70, thereby spirally advancing the second surgical component toward the distal end 80.

In the foregoing detailed description, the invention has been described with reference to specific embodiments. However, it may be appreciated that various modifications and changes can be made without departing from the scope of the invention as set forth in the appended claims. Thus, some of the features of preferred embodiments described herein are not necessarily included in preferred embodiments of the invention which are intended for alternative uses.

1. An internally threaded tube, comprising:
(a) a tube casing having (i) an inner surface with a substantially circular cross-sectional profile and (ii) an outer surface, wherein a ratio of the length of the tube casing to the inner diameter of the tube casing is greater than 5:1; and
(b) a coil positioned coaxially within the tube casing, the coil comprising an elongate element formed into a generally helical shape, with a first portion of the element interfacing with the inner surface of the tube casing and a second portion of the element projecting inwardly to form internal threads, wherein the first portion of the element is specially adapted to be bonded to the tube casing and the coil is bonded to the tube casing at one or more sites along the interface of the first portion of the element and the inner surface of the tube casing.

2. The internally threaded tube of claim 1, wherein the coil exerts a radially outward force on the inner surface of the tube casing.

3. The internally threaded tube of claim 1, wherein the element of the coil is generally cylindrical, and wherein the first portion of the element being specially adapted to be bonded to the tube casing comprises a cross-sectional profile of the first portion of the element being less curved than a cross-sectional profile of the second portion of the element.

4. The internally threaded tube of claim 1, wherein the first portion of the element being specially adapted to be bonded to the tube casing comprises the coil being centerless ground so that the interface of the first portion of the element and the inner surface of the tube casing has increased surface contact, as compared with a similar coil that is not centerless ground.

5. The internally threaded tube of claim 1, wherein the coil is laser welded to the tube casing at one or more sites along the interface of the first portion of the element and the inner surface of the tube casing.

6. The internally threaded tube of claim 5, wherein a first site is on a first coil revolution, a second site is on a ninth coil revolution, and a third site is on a seventeenth coil revolution, with second through eighth coil revolutions and tenth through sixteenth coil revolutions being un-bonded.

7. The internally threaded tube of claim 1, wherein the outer surface of the tube casing has a substantially circular cross-sectional profile.

8. The internally threaded tube of claim 1, wherein the coil has a pitch of approximately 1/24 inch.

9. The internally threaded tube of claim 1, wherein the internal threads are adapted to mate with a threaded object having a minor diameter of approximately 0.19 inches.

10. A method of creating an internally threaded tube, comprising:
(a) providing a tube casing having (i) an inner surface with a substantially circular cross-sectional profile and (ii) an outer surface, wherein a ratio of the length of the tube casing to the inner diameter of the tube casing is greater than 5:1;
(b) providing a coil comprising an elongate element formed into a generally helical shape with a first portion of the element being specially adapted to be bonded to the tube casing;
(c) positioning the coil coaxially within the tube casing, the first portion of the element interfacing with the inner surface of the tube casing; and
(d) bonding the coil to the tube casing at one or more sites along the interface of the first portion of the element and the inner surface of the tube casing, a second portion of the element projecting inwardly to form internal threads.

11. The method of claim 10, wherein the coil has an outer diameter that is equal to or greater than the inner diameter of the tube casing, and the coil exerts a radially outward force on the inner surface of the tube casing when the coil is positioned coaxially within the tube casing.

12. The method of claim 10, wherein the element of the coil is generally cylindrical, and wherein the first portion of the element being specially adapted to be bonded to the tube casing comprises a cross-sectional profile of the first portion of the element being less curved than a cross-sectional profile of the second portion of the element.

13. The method of claim 10, wherein the first portion of the element being specially adapted to be bonded to the tube casing comprises the coil being centerless ground so that the interface of the first portion of the element and the inner surface of the tube casing has increased surface contact, as compared with a similar coil that is not centerless ground.

14. The method of claim 10, wherein bonding comprises laser welding.

15. The method of claim 14, wherein bonding comprises:
(i) positioning a laser welder proximate to the outer surface of the tube casing,
(ii) laser welding a first coil revolution of the coil to the tube casing at a first site,
(iii) translating the laser welder longitudinally along the outer surface of the tube casing past a first predetermined number of coil revolutions,
(iv) laser welding a second coil revolution of the coil to the tube casing at a second site,
(v) translating the laser welder longitudinally along the outer surface of the tube casing past a second predetermined number of coil revolutions, and
(vi) laser welding a third coil revolution of the coil to the tube casing at a third site.

16. The method of claim 15, wherein the first and second predetermined number of coil revolutions is seven.

17. The method of claim 14, wherein laser welding comprises subjecting the outer surface of the tube casing to a laser weld with a laser having a focal point diameter approximately 0.003 inches less than the width of the interface of the first portion of the element and the inner surface of the tube casing.

18. The method of claim 10, wherein bonding comprises directing a high-energy beam from the outer surface of the tube casing radially inwardly to bond selected individual coil revolutions to the tube casing.

19. The method of claim 10, further comprising:
(e) positioning a fixture coaxially within the coil, the fixture having threads with a pitch that is complementary with a pitch of the coil; and
(f) positioning both the coil and the fixture coaxially within the tube casing.

20. The method of claim 19, wherein threads of the fixture are deep enough to permit the coil to deflect radially inwardly while the coil and the fixture are being positioned coaxially within the tube casing.

21. The method of claim 19, wherein bonding comprises programming a laser welder to laser weld the coil to the tube casing based on the pitch of the threads of the fixture.

22. A method of spirally delivering a surgical component to internal tissue comprising:
(a) providing an internally threaded tube that includes:
(i) a tube casing having (A) an inner surface with a substantially circular cross-sectional profile and (B) an outer surface, wherein a ratio of the length of the tube casing to the inner diameter of the tube casing is greater than 5:1, and
(ii) a coil positioned coaxially within the tube casing, the coil comprising an elongate element formed into a generally helical shape, with a first portion of the element interfacing with the inner surface of the tube casing and a second portion of the element projecting inwardly to form internal threads, wherein the first portion of the element is specially adapted to be bonded to the tube casing and the coil is bonded to the tube casing at one or more sites along the interface of the first portion of the element and the inner surface of the tube casing;
(b) positioning a distal end of the internally threaded tube proximate to internal tissue; and
(c) spirally delivering a surgical component from a proximal end of the internally threaded tube through the distal end of the internally threaded tube to the internal tissue.

23. The method of claim 22, wherein the coil exerts a radially outward force on the inner surface of the tube casing.

24. The method of claim 22, further comprising spirally loading the internally threaded tube with a plurality of surgical components, wherein spirally delivering a surgical component comprises spirally driving a surgical component nearest the proximal end of the internally threaded tube with an instrument, thereby causing a surgical component nearest the distal end of the internally threaded tube to be spirally delivered to the internal tissue.

25. The method of claim 22, wherein the internal tissue is hard tissue.

26. The method of claim 22, wherein the internal tissue is soft tissue.
